(19) United States
(12) Patent Application Publication
(10) Pub. No.: US 2026/0240583 A1
(43) Pub. Date: Aug. 20, 2026

(54) L-SHAPED TOOL FOR A SET FOR TREATING BONE FRACTURES

(71) Applicant: I.T.S. GmbH, Lassnitzhöhe (AT)

(72) Inventors: Wolfgang AUF, Hart bei Graz (AT); Thomas RESEDARITZ, Großhartmannsdorf (AT); Ronald PRAGER, Bovenau (DE)

(73) Assignee: I.T.S. GmbH, Lassnitzhöhe (AT)

(21) Appl. No.: 19/544,452

(22) Filed: Feb. 19, 2026

(30) Foreign Application Priority Data
Feb. 20, 2025 (AT) A 50120/2025

(51) Int. Cl. A61B 17/88; A61B 17/72; A61B 17/00; A61B 17/56

(52) U.S. Cl. CPC A61B 17/8875; A61B 17/725; A61B 2017/00424; A61B 2017/564

(57) ABSTRACT

A tool for a set for treating bone fractures, in particular proximal femur fractures, a set for treating bone fractures and a method for treating bone fractures. The tool includes a base body having a first, in particular proximal, end and a second, in particular distal, end; a grip region; and a connector for positioning the tool on a part of the set, in particular a bone screw. The connector has a groove.

### CROSS-REFERENCE TO RELATED APPLICATION

This application claims priority under 35 U.S.C. § 119(a) to Austria Application No. A 50120/2025 filed February 20, 2025, the disclosure of which is expressly incorporated by reference herein in its entirety.

### BACKGROUND

### FIELD OF THE INVENTION

Embodiments relates to a tool for a set for treating bone fractures, in particular proximal femur fractures, that includes a base body having a first, in particular proximal, end and a second, in particular distal, end and a grip region and a connecting region for positioning the tool on a part of the set, in particular a bone screw.

Embodiments also relate to a set for treating bone fractures, in particular proximal femur fractures, that include a bone nail, a bone screw, and a set screw, in which the bone nail includes an opening through which the bone screw is guided, and the set screw is configured to be brought into contact with the bone screw in order to define at least one movement possibility of the bone screw.

Embodiments also relate to a method for treating bone fractures, in particular proximal femur fractures, in particular using a set of the type named at the outset and/or involving the use of a tool of the aforementioned type, in which a bone screw and a set screw which interacts with the bone screw are guided through an opening of a bone nail arranged in a bone piece, so that the set screw runs obliquely to the bone screw. The set screw is configured to be brought into contact with the bone screw in order to define at least one movement possibility of the bone screw.

### DISCUSSION OF BACKGROUND INFORMATION

Femur fractures, in particular in the proximal region, require careful treatment. Fractures of this type, such as femoral neck fractures, occur primarily in older people. Since a bone substance is often no longer in an ideal condition in older people, operations to treat femur fractures, in particular in the proximal region, can prove to be challenging. It is thus even more important to enable a surgical process to be as simple as possible for a surgeon, yet also with sufficient options for individual adaptation of the implant introduced for the treatment. Unforeseen complications during the introduction of the implant can result in the need to reverse and restart individual steps again in the step-by-step sequence of introducing the implant, which not only prolongs a length of surgery, but also leads to a mental strain on the surgeon and, due to additional process steps, naturally also increases the risk of complications. In this respect, a certain conflict of objectives is present, since instruments for the introduction of implants for treating, in particular proximal, femur fractures should, on the one hand, be designed to be as simple as possible in order to avoid complications during the setting of the implant, but, on the other hand, should offer the greatest possible flexibility during the operation despite the simple usability.

From the prior art, diverse sets for the treatment of, in particular proximal, femur fractions have become known. Sets which, in the treatment of proximal femur fractures, comprise, in addition to a bone nail that is to be introduced, which is inserted into the femoral neck, a bone screw as well as a further screw for fixing the bone screw are presently considered to be at the technologically forefront. In this at least three-piece set, the bone nail or intramedullary nail comprises an opening through which the bone screw is guided transversely through the bone nail in order to engage in a femoral head. The bone screw is thereby situated in the opening such that the bone screw can slide. In order to limit a movement of the bone screw, for example a rotation thereof or a movement of the bone screw in a lateral direction, an additional set screw is provided with which the bone screw interacts. The set screw can be guided through the same opening as the bone screw or, if necessary, also a separate opening of the bone nail, but is fixed to the bone nail via an internal thread in the opening.

Multi-piece sets for the treatment of, in particular proximal, femur fractions can be designed, for example according to WO 2024/098081 A1 from the Applicant, such that the set screw runs through an opening of the bone nail, through which opening the bone screw is also guided. The set screw is arranged such that it runs obliquely to the bone screw and interacts therewith. For this purpose, multiple grooves that conically taper can be formed on the outside of the bone screw, which is in principle cylindrically shaped. Depending on the positioning, the obliquely positioned set screw, which is fixed by a thread, engages in said conically tapered grooves. The bone screw can then move in a lateral direction until the conically tapered groove is blocked by the fixed set screw. At the same time, a mobility of the bone screw in terms of a rotation, and therefore also a twisting of the femoral head, is prevented, since the groove of the bone screw, and therefore also the bone screw, cannot be rotated about the set screw which engages in the contact volume. Sets of this type have also been further developed such that, during an operation, an apposition or compression of the separated bone pieces that are to be treated can take place using additional instruments if necessary.

Although sets such as that known from WO 2024/098081 A1, if necessary together with additional instruments for an apposition and/or compressions, are already relatively easy to use and offer many possibilities, it has nevertheless been shown that, during an operation, it can occur that in a, when the set screw is introduced, the already-positioned bone screw is located in a not entirely correct position, despite prior alignment using markings, for example. This can result in an inability to initially guide a drill, with which a borehole for the set screw is to be drilled, past the bone screw. Even if it is still possible to introduce a borehole, an incorrect positional alignment of the bone screw can cause the inability of the set screw to engage, or to come to rest with a medial end, in that volume which is formed by a groove of the bone screw. In such situations, in the most complicated case, the bone screw must be removed again and the process must be started over again. With the given bone material, this is not only disadvantageous, but also challenging for a surgical team, and also increases the risk of potential complications due to a non-optimal treatment of the fracture. Even in the case of a more simple rectification of the problem, when the set screw has been set, at least the set-screw driver, together with the guide sleeve, must be removed again in order to suitably adjust the bone screw, which does not necessarily lead to success, however.

### SUMMARY

Embodiments are directed to a tool of the type named at the outset which allows, in a simple manner, the bone screw to be readjusted without removing the bone screw prior to the insertion of the set screw, if it should be necessary to do so.

According to embodiments, a readjustment of an already-attached bone screw, in particular when attached to a femoral head, of a set of the type named at the outset is permitted in a simple manner.

Other embodiments are directed to a method for treating bone fractures, in particular proximal femur fractures, in which a bone screw and a set screw which interacts with the bone screw are guided through an opening of a bone nail arranged in a bone piece so that the set screw runs obliquely to the bone screw. The set screw is configured to be brought into contact with the bone screw in order to define at least one movement possibility of the bone screw, such that, once the bone screw has been attached to a bone piece, in particular a femoral head, the bone screw can, in a simple and most reliable possible manner for a surgical team, be brought into a correct position for the introduction of the set screw.

Aspects of the claimed embodiments are achieved, in the case of a tool of the type named at the outset, if the connecting region includes a groove.

With a tool according to embodiments, it may be advantageous if the tool enables, in a simple manner, a correct alignment of the bone screw in relation to the set screw, which only later runs (slightly) obliquely to the bone screw. In principle, the bone screw is, after the positioning thereof, in particular in a femoral head, already aligned such that the set screw, which is to be introduced slightly obliquely to a longitudinal axis of the bone screw, can suitably interact with the bone screw. For an arrangement of the bone screw and of the set screw, suitable guide sleeves are introduced into what is referred to as a master sleeve, in which tissue protection sleeves are already positioned. Depending on the course of the operation, suitable guide sleeves, for example, first for drill wires and then later for the bone screw, as well as a drill for drilling a hole for the set screw, are introduced into the master sleeve together with tissue protection sleeves. Once the bone screw has been guided and introduced via the drill wire for the bone screw, it can occur, for example as a result of a repositioning of the patient or other processes, that individual sleeves shift and/or twist, since a certain tolerance of a few degrees (typically less than 5°) is provided for said sleeves. However, this can already be enough that the bone screw is no longer correctly aligned in relation to a sleeve for the drill for the set screw. If the drill is affected, this can cause an inability to guide the drill past the bone screw. However, even if the drill can still be guided past, it can subsequently occur that the set screw already unintentionally strikes the bone screw in a lateral region and cannot, as intended, engage in a contact volume that is formed by one or more externally arranged, preferably equidistantly arranged on the circumference, grooves of the bone screw. As a result of the tool, it is now possible to easily move, in particular to rotate, the bone screw, with said bone screw remaining attached to the bone piece such as a femoral head. This can occur until the drill or the set screw can enter the groove defined by the tool, which groove lies distally to the bone screw, the groove of the tool that is distally freely accessible. The tool is thereby configured such that said tool grasps the bone screw in such a manner that, with the positioning of the tool on the bone screw, the position of the at least one groove of the bone screw (or, where applicable, another type of an interacting region such as a stop) is also fixed relative to the tool. It is thus ensured that, with the entry of the drill or of the set screw into the distally arranged groove of the tool, the correct positioning in relation to the external groove of the bone screw is also provided. By this constructionally simple measure, the bone screw can be correctly aligned by an easy manual movement with the tool, if, despite a previously created correct alignment of the markings on a tissue protection sleeve and the bone screw and/or a screwdriver for the bone screw, a blockage for the drill and/or the set screw arises. In addition, the tool can, despite the constricted spatial conditions for the introduction of two interacting screws (bone screw and set screw), remain fastened to the bone screw during further operation, in particular a drilling for the set screw and the introduction of the set screw. This also allows the provision of additional functionality on the tool, such as that which is explained further below.

A fastening of the tool to the bone screw can, for example, take place via a medial projection which engages in a lateral end of the bone screw in a form fit. As a result, in particular a groove of the connecting region can be fixed relative to a groove on the bone screw, since only predefined positions can be realized depending on the symmetry of the form fit. Additionally, inside of the connecting region, a connectivity means can be provided for the releasable lateral fixing of the bone screw. The connectivity means can, in particular, be a hollow rod that can be guided through the connecting region. At a medial end, said rod can comprise a thread, in particular an external thread, which interacts with a lateral thread of the bone screw, in particular an internal thread. At a lateral end, the connectivity means can comprise a widened end with which the connectivity means, such as a rod, can be rotated in order to produce a connection to the bone screw.

The groove is preferably embodied such that it runs in a longitudinally extended manner between a lateral end and a medial end. The groove can have a cross section that is adapted to an external diameter of the drill for the set screw and the set screw and/or a guide sleeve. Normally, the groove is embodied with a cross section in the shape of a circular sector. Said cross section can be designed such that it is widened when viewed in the medial direction, since the set screw does not run parallel to the bone screw, but rather at a slightly oblique position (angle of up to 20°, preferably up to 15°, in particular 10° or less, for example less than 3°) to the bone screw. A corresponding embodiment of the groove of the tool adequately allows for said oblique position. The groove can thus run obliquely relative to a fastening axis. The fastening axis of the bone screw coincides with a longitudinal axis of the same. This results, relative to the fastening axis or longitudinal axis, in the noted angle, at which the set screw runs obliquely relative to the bone screw.

The groove in or on the connecting region can be embodied with a length which is at least one third, preferably at least half, of a length of the connecting region. The groove can, transversely to the longitudinal axis thereof, have a maximum width which is at least 60%, preferably at least 70%, in particular 80% or more, of a diameter of the connecting region transverse to a longitudinal axis of the same.

The connecting region is preferably embodied to be shaft-shaped. In terms of length, the connecting region is configured to span a distance to an already-set bone screw. Accordingly, the connecting region is preferably embodied as an elongate tool part which extends from the base body of the tool in the medial direction. In particular, the connecting region can be embodied to be roughly shaft-shaped. If necessary, the connecting region can be embodied to be hollow in the shaft-shaped portion thereof, in particular in order to insert a fastening instrument through a hollow interior of the connecting region, with which instrument the bone screw can be releasably fastened.

It is particularly preferred that, at the second end, the base body comprises a receptacle for the fitting-on and fastening of the tool to an, in particular lateral, end of a bone screw. For the sake of easier handling, the lateral fastening is preferably possible by means of an elongate shaft of the tool, which shaft constitutes the connecting region. The shaft can be permanently connected to the base body or can also be embodied as a separate part of the tool which can be releasably fastened to the base body. As explained, the tool can thereby be releasably connected to the bone screw at a lateral end of the bone screw. The receptacle can preferably be a projection that can be fitted-on at a lateral end of the bone screw. The medially arranged projection advantageously engages in a lateral end of the bone screw in a form fit, so that the medially arranged projection interacts with the lateral end of the bone screw in a form fit. To fix a connection of the tool to the bone screw in a translation direction between medial and lateral, a fastening instrument guided through the hollow shaft can be used, which instrument can be inserted at a lateral end of the tool and actuated in said position in order to also fix, in addition to the form-fitting projection, the bone screw in relation to a translation between lateral and medial. In particular, this can be the aforementioned connectivity means. In principle, however, it is also possible that a connection of the bone screw to the base body is produced via another, separate tool component.

The base body, which is typically made of a steel, in particular from a rustproof steel, can comprise an adjusting screw, wherein the adjusting screw runs parallel to the fastening axis. The adjusting screw can, for example, be embodied in the form of a grub screw. The adjusting screw is advantageously guided through the base body. It can thereby in particular be provided that the base body comprises an internal thread in which an external thread of the adjusting screw engages, wherein the adjusting screw is embodied to be longer than a distance between a lateral edge and a medial edge in the region of the adjusting screw and along the fastening axis. In other words, the adjusting screw is embodied to be longer than the noted distance between the lateral edge and the medial edge. On the basis of the internal thread in the base body and the external thread of the adjusting screw, which external tread interacts therewith, the adjusting screw can be screwed through the base body. The adjusting screw is thus movable relative to the base body, or vice versa the base body relative to the adjusting screw, when the adjusting screw bears against a stop and is actuated. Precisely this fact can be utilized in order to move the tool, together with a bone screw directly or indirectly fastened thereto, in a lateral direction when a medial end of the adjusting screw bears against a part of the set for the operation, for example a tissue protection sleeve. If the adjusting screw is then actuated, the tool, together with the bone screw, is pushed in the lateral direction. An apposition of the separated bone pieces is thus possible. A lateral maximum movement is thereby limited by an engagement of the set screw. It is thereby particularly preferred that the adjusting screw is arranged adjacently to the first end, in particular adjacently to the connecting region. The critical axes (fastening axis along the connecting region and longitudinal axis of the bone screw, and translation axis of the adjusting screw) are thus close to one another, which is beneficial for a reliable translation of the tool together with the bone screw in the lateral direction. Nevertheless, the adjusting screw could also be positioned at another position of the tool, provided that a necessary stop on a part of the set is found in order to enable the relative movement between the adjusting screw and the remaining parts of the tool with a simultaneous displacement of the latter.

In an enhancement, it can be provided that the tool comprises a locking device for the releasable fastening of the tool to a further part of the set. This can be an actuating means (actuator) for the set screw, for example a screwdriver. The actuating means typically comprises a stop against which the locking device can come to rest downstream when viewed from a medial towards a lateral direction. If the locking device is arranged after the stop in a direction from the medial towards the lateral, and if the tool is simultaneously connected to the bone screw, this initially results in a locking between the actuating device for the set screw and the bone screw, which is situated such that it can slide in the opening of the bone nail. If the set screw, the external thread of which engages in an internal thread of the opening of the bone nail, is thus loosened with the actuating device such as a screwdriver, that is, moved laterally by a rotation to the left, the stop moves the locking device of the tool, and therefore the tool and, as a result, also the bone screw, in tandem in the lateral direction. Even if the set screw itself would already prevent a further lateral movement of the bone screw, as in the case of the previously explained apposition, this is not the case in this process, since in this case the set screw is also moved in the lateral direction, as is therefore the entire combination of the bone screw and set screw. It is thus possible to press against one another, and to thus compress, the bone pieces which are separated from one another. The functionality of the tool is thus expanded once again, so that the tool is ultimately a multifunctional tool having the following capabilities:

correctly aligning the bone screw for the introduction of the set screw;

performing an apposition; and

performing a compression.

At the same time, the tool is not only embodied in a simple manner, but also easy to handle, and provides little chance for errors. The tool also does not need to be detached during the individual steps, but rather can remain fastened for the purpose of carrying out the functionalities listed above, which additionally improves usability and helps to reduce errors.

The locking device can, in particular, be arranged in the grip region. Preferably, the locking device is mounted such that it can be longitudinally displaced between the first end and second end. The tool can thereby comprise a release and fixing mechanism for the locking device. In a constructional design of this type, it is easily possible to initially release and longitudinally displace the locking device, that is, to displace it from a proximal into a distal position. In said distal position, the locking device can come to rest against the noted stop of the actuating device for the set screw. If necessary, locking is possible again in this position, should it be necessary to do so. The locking device can be subsequently released and displaced into the proximal home position again. An ability to longitudinally displace the locking device, preferably transversely to the fastening axis, and therefore a longitudinal axis of the bone screw, is thus provided and the locking device is mounted such that it can be displaced transversely to the fastening axis.

The base body is advantageously embodied to be essentially flat. Essentially flat thereby means that the base body can be manually grasped, but the embodiment along the extension from the first end to the second end is essentially provided with an equal thickness in plan view. The base body is thus easy to handle, offers the desired and previously explained functionalities, and nevertheless does not constitute a hindrance during surgical steps that take place after the fastening of the tool. A shaft can be arranged on the base body in the region of the second, in particular distal, end, which shaft can interact with the bone screw, in particular with the lateral end of the bone screw. For this purpose, an external thread can be provided on the shaft at a free, medially engaging end, which external thread interacts with a lateral inner thread of the bone screw at the end.

The tool is preferably also used to screw-in the bone screw itself, and thus also takes on the function of a screwdriver.

Aspects of other embodiments are achieved by a set of the type named at the outset if the set comprises a tool for the, in particular rotating, movement of the bone screw in a fastened state, in particular a tool according to the invention as explained above, wherein the tool comprises a groove for the set screw.

With a set of this type, surgical tools for introducing an implant in the form of a bone nail and a bone screw interacting therewith and a set screw can be provided, which surgical tools prove to be advantageous for a most error-free possible operation with quick positioning of the bone screw in a correct position for accommodating the set screw. When configured according to the embodiments, an apposition (closer positioning of the surfaces of broken bone pieces to one another) can, if necessary, also take place using the set and, where required, also a compression (pressing against one another of bone pieces that have separated from one another). The tool can thereby remain fastened to the bone screw from the step of a possible repositioning of the bone screw prior to the introduction of the set screw and an apposition, up to a compression. This is noteworthy because the spatial conditions themselves in the region of the lateral receptacle of the master sleeve, via which both the bone screw and the slightly obliquely positioned adjusting screw are introduced, are very limited.

The set screw can, as mentioned, be fixable in the bone nail such that the set screw runs at an angle oblique to the bone screw. For an oblique positioning, it is sufficient if the angle is relatively small. Angles of < 10°, preferably < 5°, in particular < 3°, are normally sufficient.

The set screw is configured to engage in a contact volume defined by the bone screw. A contact volume can be predefined by one or more grooves on the bone screw. The groove or the grooves are thereby arranged externally on the bone screw. The bone screw is embodied to be essentially cylindrical. The cylindrical embodiment can be present with a circular cross section. The cylindrical embodiment of the bone screw occurs with a longitudinal extension along a longitudinal axis of the bone screw. At a medial end, the bone screw comprises an external thread which engages in a bone piece. At a lateral end, the bone screw normally comprises a geometric structure, arranged in a recessed manner, for the insertion of a screwdriver or of another actuating means with which the bone screw can be set in rotation about the longitudinal axis thereof, so that the bone screw can be screwed into a bone piece once a borehole has been set. At the lateral end, the bone screw can furthermore have an asymmetrical shape which differs from the cylinder shape so that, at this position, the tool can only be fitted-on using the receptacle in a single position. As a result, incorrect mounting of the tool on the bone screw is avoided and a relative alignment of the groove or grooves of the bone screw relative to the tool is instantly provided. The groove or the grooves define one or more contact volumes in which the set screw can engage. Because the set screw engages in particular with a medial end in a volume which is formed by a groove, a significant rotation of the bone screw and femoral head is initially prevented, since the groove then strikes a medial end of the set screw. A movement of the bone screw within the meaning of a rotation of the same about a rotation axis different from the longitudinal axis of the bone screw is thus prevented. To the extent that a lateral movement of the bone screw is concerned, said movement can likewise be either completely prevented or allowed in predetermined amounts by the set screw. Since the groove or the grooves on the bone screw are typically embodied to be conically tapered, namely from a lateral side towards a medial side, the bone screw can, with a suitable adjustment of the set screw, continue to move laterally until the groove of the bone screw, which groove becomes increasingly conically narrow, is ultimately blocked by the set screw, which prevents a further lateral movement of the bone screw. However, it is also possible that a lateral displacement of the bone screw is entirely prevented during the healing process in that the set screw is immediately positioned at the stop. In addition to the embodiment with a conical groove of the bone screw, however, other variants are also used which enable specific lateral movements of the bone screw, for example a tiered embodiment of a groove. In the contact volume, the set screw does not necessarily need to initially touch the bone screw; rather, it is sufficient that the set screw, in particular a medial end of the same, protrudes into said volume.

The set according to embodiments can additionally comprise drill wires, a master sleeve, and one or more tissue protection sleeves, diverse guide sleeves, a targeting device for the bone nail, and equipment such as screwdrivers.

Other aspects of the embodiments of the method can be achieved when, the bone screw is moved using a tool, in particular a tool according to the invention, for the purpose of positioning the set screw.

In the method according to the embodiments, it is advantageous that, in a simple manner, the bone screw can be easily, quickly, and accurately brought into a correct position in the event of a possible shifting of a tissue protection sleeve, and therefore of the marking necessary for the correct alignment of the bone screw itself, so that both the drill for the set screw and the set screw itself can be correctly inserted. Within the scope of the method, a set according to the invention is thereby advantageously used, which set, in turn, advantageously comprises a tool according to the invention.

Within the scope of the method, it can in particular be provided that the bone screw is essentially moved by rotation using the tool. During the screwing-in, a translation transverse to a longitudinal axis of the bone screw can also occur.

According to the method, the bone screw can be grasped using the tool and be moved in a lateral direction using an adjusting screw of the tool. As a result, an apposition is possible while the set screw is simultaneously stationary.

Furthermore, within the scope of the method, the bone screw may be grasped with the tool and the tool can be placed against a stop connected to the set screw or formed by said set screw itself, whereupon the bone screw is moved by the tool in that, due to a medial stop of a setting means of the tool, such as an adjusting screw, a part of the tool, together with the bone screw connected thereto, is displaced in the lateral direction. A compression, that is, a pressing against one another of bone pieces separated from one another, is thus possible.

Other exemplary embodiments and advantages of the present invention may be ascertained by reviewing the present disclosure and the accompanying drawing.

### BRIEF DESCRIPTION OF THE DRAWINGS

Additional features, advantages, and effects of the invention follow from the exemplary embodiments described below, as well as the explanations regarding the performance of an operation using a tool according to the invention as well as a set in which the tool is used. In the drawings which are thereby referenced:

FIG. 1 shows a side view of a tool according to the invention, fastened to a bone screw;

FIG. 2 shows a frontal view of the tool according to FIG. 1 together with a bone screw;

FIG. 3 shows a plan view of the tool together with the bone screw according to FIG. 1;

FIG. 4 shows a rear view of the tool together with the bone screw according to FIG. 1;

FIG. 5 shows a bottom view of the tool together with the bone screw according to FIG. 1;

FIG. 6 shows the tool according to FIGS. 1 through 5 in a perspective view, but without a bone screw;

FIGS. 7 through 11 show the situation according to FIGS. 1 through 5 during an operation step;

FIG. 12 shows the tool according to FIGS. 7 through 11 in a perspective illustration;

FIG. 13 shows a detail from a set for treating a proximal femur fracture with a fastened tool;

FIG. 14 shows the set according to FIG. 13 during the performance of an apposition of separated bone pieces;

FIG. 15 shows the set from FIGS. 13 and 14 during the performance of a compression of separated bone pieces;

FIG. 16 shows a perspective view of a set during an operation step.

FIG. 17 shows an arrangement of a bone screw and a set screw extending through a bone nail.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

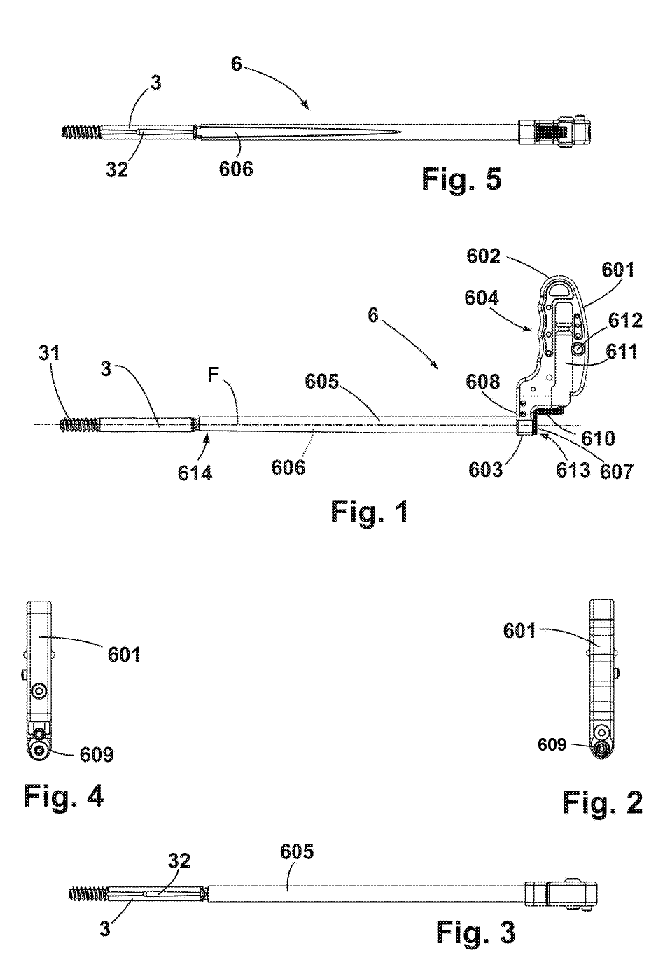

In FIGS. 1 through 4, a tool 6 according to embodiments is illustrated. As can be seen in FIG. 1, the tool 6 is arranged on a bone screw 3, the function of which will be explained further below. The bone screw 3 is embodied to be essentially cylindrical and elongate, and has a longitudinal axis which coincides with a fastening axis F. The bone screw 3 is configured to be introduced into a bone piece or fixed in said bone piece, in particular a femoral head, and comprises an external thread 31 at a medial end for this purpose.

As can be seen from FIGS. 1 through 5, the tool 6 comprises a base body 601 that can be made of a steel, in particular, a rustproof steel. The base body 601 comprises a first end 602 and a second end 603. Based on the roughly elongate embodiment of the base body 601 from top to bottom in the depiction in FIG. 1, the second end 603 lies opposite from the first end 602. The first end 602 can be considered to be a first, proximal end 602 and the opposite, second end 603 a second, distal end 603. The base body 601 is, as can be seen in particular from FIG. 2, but also from considering FIGS. 1 through 5 together, embodied to be essentially flat. A certain thickness results from the requirement that the base body 601 should be easy to grasp for surgical personnel. For this purpose, the base body 601 comprises a grip region 604 which is arranged adjacently to the first end 602, or connects thereto, and extends in a distal direction towards the second end 603. In the grip region 604, the tool 6 is embodied not only with an essentially constant thickness (as in the remaining regions as well), but also essentially with a constant width, as can be seen in the side view in FIG. 1. On a medial front face of the grip region 604, a wave shape is provided which facilitates a manual grasping in the grip region 604 by a human hand, and therefore a secure hold of the tool 6 for surgical personnel. Connecting to the grip region 604 is a connecting region (connector) 605 which comprises at the second end 603 a receptacle 609 that can be seen in FIGS. 2 and 4. Receptacle 609 is embodied such that it accommodates the connecting region 605. The corresponding connection between the base body 601 and connecting region 605 can be releasable or permanent. Via a medial end of the connecting region 605, the tool 6 can be slid onto a lateral end of the bone screw 3 in a form fit. Since the bone screw 3 is, at the lateral end thereof, embodied with a recess having a symmetry corresponding to a multi-point rotation axis (for example, a polygon such as a triangle or pentagon or hexagon) and the medial end of the connecting region 605 is shaped such that it corresponds thereto, there is preferably only the possibility of connecting the connecting region 605 to the lateral end of the bone screw 3 such that false arrangements of the tool 6 on the bone screw 3 cannot occur. Advantageously, the receptacle 609 extends as a continuous opening between a lateral edge 607 and a medial edge 608 of the tool 6, which accommodates the connecting region 605, which is embodied to be essentially shaft-shaped or as a shaft. As can be seen in the side view in FIG. 1, the tool 6 is thereby embodied to be considerably longer in the connecting region 605 compared to the grip region 604. In particular, the connecting region 605 is embodied to be so long that the bone screw 3 can be attached to a bone in a desired manner and can be manipulated using the tool 6. Furthermore, the grip region 604 is embodied to be laterally recessed relative to the connecting region 605. It is thus possible to be able to fasten the tool 6 to the bone screw 3 with good visual contact, even if the tool 6 is grasped by a thick hand. In other words, it can also be stated that the connecting region 605 is embodied to be protruding relative to the grip region 604. With the grip region 604 and the relatively wide embodiment thereof, and the connecting region 605 embodied to be considerably longer, a sufficiently good force transfer is also enabled, so that a rotation of the tool 6, and therefore of the bone screw 3 connected thereto, is possible with a small application of manual force.

A further component of the tool 6 is an adjusting screw 610, which in particular can be embodied as a grub screw. The adjusting screw 610 is embodied to be longitudinally extended. The adjusting screw 610 comprises an external thread which interacts with an internal thread that runs parallel to the fastening axis F, and therefore also to a longitudinal axis of the bone screw 3 and of the fastening axis F.

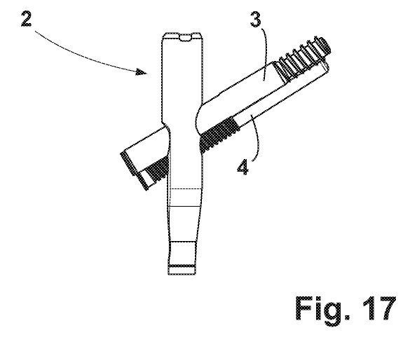

Furthermore, in a region of the connecting region 605, the tool 6 comprises a lateral end 613 and a medial end 614 and, adjacently to the medial end 614, a groove 606 which preferably starts from said end, which groove 606 can in particular be seen in FIG. 5. Upon a first consideration, the distally arranged groove 606 is used to clear the way for a set screw (not shown) that is to be inserted later or, already prior to this, to clear a path for a drill for the purpose of introducing a hole for the set screw which interacts with the bone screw 3. In a second consideration, which is based on the first consideration, a correct entry of the set screw can also be ensured with the explained form-fitting connection of the tool 6 to the lateral end of the bone screw 3. Through a single possible form-fitting connection of the tool 6 to the bone screw 3 at the lateral end thereof, it namely occurs that the position of the groove 606 of the bone screw 3 can also be set using the tool 6. If the coordination is such that the groove 606 of the tool 6 also enables an entry by the set screw, and before that by the drill for the same, a correct positioning of the bone screw 3 and of one of possibly multiple external grooves 32 is automatically provided, in which groove 32 of the bone screw 3 the set screw 4 comes to rest in a contact volume, in particular with a medial end of the set screw 4, see FIG. 17, where, if necessary, as a result of contact with an outside of the bone screw 3, a blockage of a rotation of the bone screw 3 about a longitudinal axis different from the longitudinal axis of the bone screw 3 and/or a lateral movement of the bone screw 3 is limited to a specific maximum amount or entirely prevented.

The tool 6 thus provides, in a first function, a tool 6 which enables a correct alignment of the bone screw 3 prior to the introduction of the set screw. This can in particular be or become important because, in any event, the bone screw 3 may not be correctly positioned due to a repositioning of a patient or other processes. Typically, a marking is affixed to a tissue protection sleeve 9, likewise to the bone screw 3 and/or a screwdriver for introducing the bone screw 3. The marking on the bone screw 3, and if necessary the extension thereof on the tool 6, are brought into correct agreement with the marking on the tissue protection sleeve 9. Because this takes place by rotating the bone screw 3, in the case of proper agreement of the markings, the bone screw 3 should be aligned with sufficient precision for the accommodation or engagement of the set screw. It can occur, however, that, during an operation, the tissue protection sleeve 9 in which the bone screw 3 runs, is twisted a few degrees, since a certain tolerance is provided for the arrangement of the tissue protection sleeve 9 in a master sleeve 8. Although said tolerance is relatively small at, for example, maximally 5°, this can nevertheless have significant consequences, since an oblique positioning of the set screw has an even smaller angle of, for example, less than 1.5°, which can result in an undesired blockage or a contact of the set screw outside or not in the desired region of the contact volume. This leads to complications during an operation. Up until now, a rectification of such situations mostly involved dismantling the system, that is, removing and repositioning the bone screw 3 again in the worst case. Now, with the tool 6 according to the invention, the bone screw 3 can, in a simple manner, be slightly rotated until the set screw, or if necessary the drill for the same already prior to that, can freely pass through the groove 606 of the tool 6, and the correct alignment of an external groove 32 of the bone screw 3 is thus ensured at the same time. An immediate correction is thus possible if complications unexpectedly arise during the introduction of the set screw.

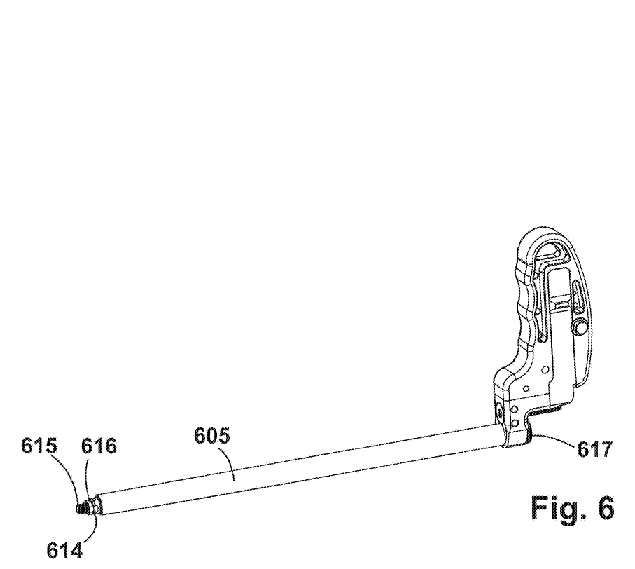

The bone screw 3 can be releasably fixedly fastened to the tool 6. For this purpose, according to FIG. 6, a connectivity means (connector piece) such as a hollow rod can be guided inside of the hollow shaft or connecting region 605. The connectivity means can, at a medial end, comprise an external thread 615 which engages in an internal thread of the bone screw 3 that is laterally arranged on the bone screw 3. Via the structure 616 arranged at the medial end 614 of the connecting region 605, a form fit of the connecting region 605 with the bone screw 3 can be achieved, so that the groove 606 is aligned with one of the grooves 32 of the bone screw 3. The releasable connection to the bone screw 3 then takes place via the connectivity means guided through the connecting region 605. For this purpose, an actuating wheel 617 can be provided on the connectivity means at the lateral end.

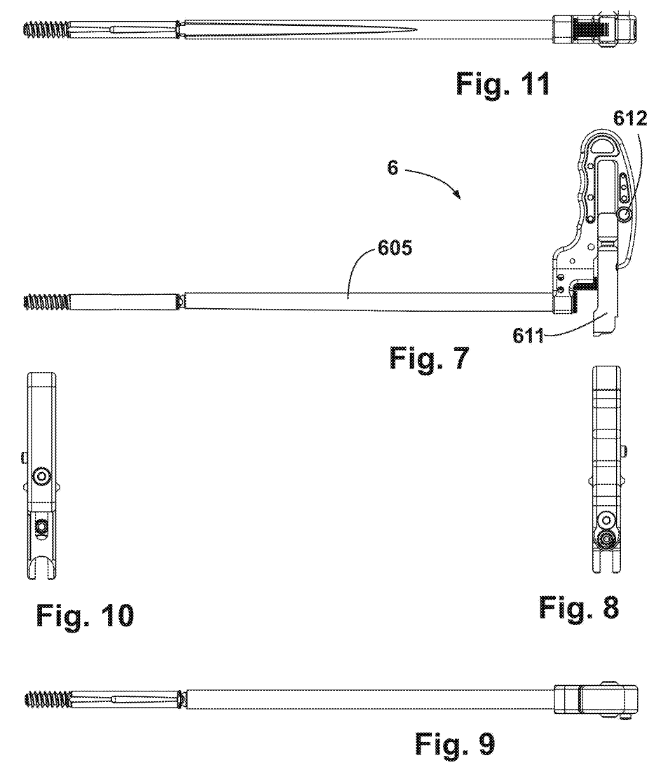

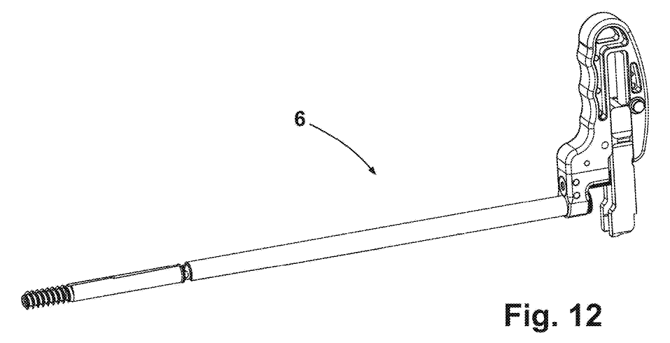

With the adjusting screw 610 and a locking device 611, visible in FIG. 1, additional functional capabilities can be created. As can be seen in FIG. 1, the locking device 611 is embodied as a longitudinal slider. The longitudinal slider interacts with a release and fixing mechanism 612. The slider can, as in the case of the base body 601, be made of a metal or an alloy, in particular in order to generate a maximum rigidity. The release and fixing mechanism 612 can, for example, constitute a typical mechanism which enables the actuation of an element that is mounted such that it can slide, when a predetermined spring force is overcome. Other, simple locking mechanisms are, of course, also possible, provided that it is ensured that the slider is initially held in a home position according to FIG. 1 through FIG. 6, but can be slid from the home position into a working position when the release and fixing mechanism 612 is actuated, which working position can be seen in FIG. 7 through FIG. 12, or automatically springs, for example in the case of a spring-loaded mount which is released, into said working position. The release and fixing mechanism 612 can be embodied such that, in said working position, the elongate slider is likewise fixed or can be fixed; however, this is not necessary.

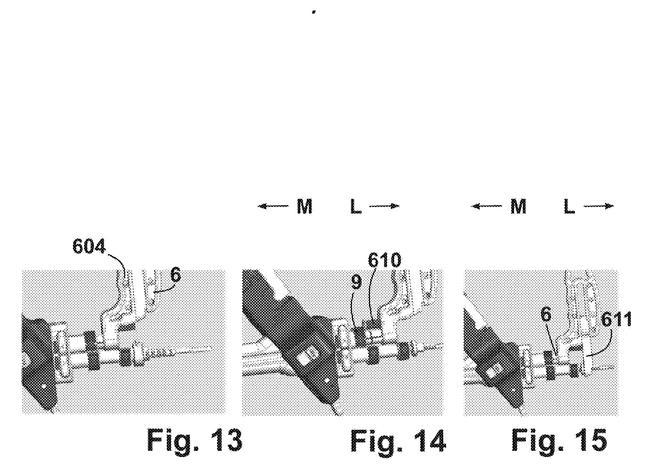

The additional functionalities obtained with the adjusting screw 610 and the locking device 611 are illustrated with the aid of FIGS. 13 through 15. In FIG. 13, a situation is shown in which a bone screw 3 is already attached to a bone piece such as a femoral head, for example. The bone screw 3 is superiorly arranged. Furthermore, the tool 6 is accommodated in a form fit on the bone screw 3 at the lateral end thereof or vice versa . Inferiorly, a guide sleeve is visible through which a drill is guided. As can be seen, the guide sleeve can, despite the constricted spatial conditions, still be guided past, even if the tool 6 is fastened to the bone screw 3 and the set screw, together with the guide sleeve therefore, enters slightly obliquely to the longitudinal axis of the bone screw 3, and therefore also to the fastening axis F. This is also possible because the base body 601 is embodied in the explained manner with a relatively narrow distal end 603 and receptacle 609 and widens towards the first end 602 with the grip region 604. However, only when the groove 606 of the tool 6 is aligned such that the set screw or the guide sleeve thereof can be guided past, is a correct alignment of the bone screw 3 present, whereby the first functionality results.

A second functionality is enabled by the adjusting screw 610, as can be seen in FIG. 14. If the tool 6 touches the bone screw 3 and, at the same time, the tool 6 bears against an end formed, for example, by a guide sleeve for the set screw, a screwing-in of the adjusting screw 610 causes a lateral displacement of the bone screw 3 once the adjusting screw 610 medially strikes, for example, a tissue protection sleeve 9, as can be seen in FIG. 14. Thus, if a medial stop is superiorly present (superior tissue protection sleeve), a screwing-in of the adjusting screw 610 when a medial stop is present results in a movement of the bone screw 3 together with the bone piece connected thereto. A closer positioning of the separated bone pieces, that is, an apposition, thus occurs. Said apposition is limited by a clearance that is predefined by the set screw or is set using said set screw. This is a second functionality, which is obtained if the adjusting screw 610 is provided.

A third functionality is explained with the aid of FIG. 15. This functionality results from the locking device 611. If the locking device 611 is actuated and linearly displaced, that is, from a proximal home position into a distal working position, a screwdriver for the set screw, for example, can thus be grasped, as can be seen in FIG. 15. Since the screwdriver for the set screw is connected to the same, but at the same time also comprises a stop, the connection via the tool 6 leads, when the screwdriver is actuated counterclockwise, to an unscrewing of the set screw, and thus also, however, to a lateral movement of the bone screw 3, wherein said lateral movement is not limited any further. Thus, in principle, any desired amount of pressure can be applied in a contact region to bone pieces separated from one another, that is, a compression. The tool 6 thus enables a correct positioning of the bone screw 3 when said bone screw 3 is incorrectly aligned, an apposition of bone pieces, and a compression of bone pieces. This is a third functionality, which is obtained if the locking device 611 is provided.

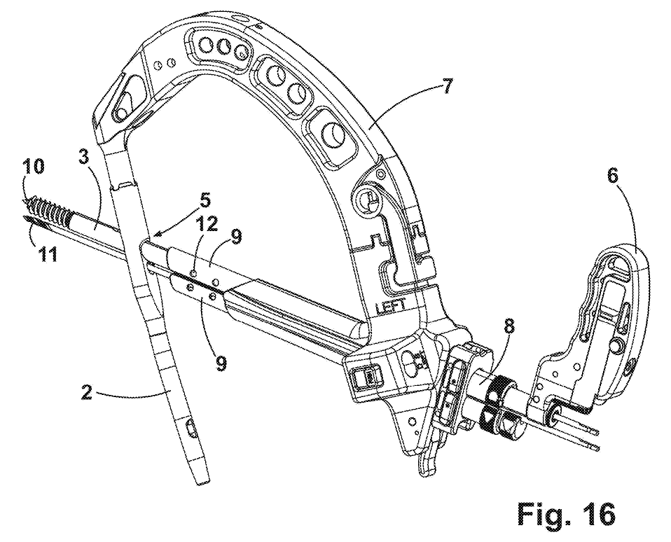

FIG. 16 shows the tool 6 during an operation step. An operation method can, in particular, proceed with the following method: Initially, in a first step, a bone nail 2 is introduced into a medullary space of a femur using a targeting device 7. The bone nail 2 can thereby also be gently hammered-in. The bone nail 2 comprises an opening 5 that is continuous and lies closer to a proximal end of the bone nail 2 than to a distal end. The opening 5 provides space for accommodating and feeding-through a bone screw 3 as well as a set screw 4, see FIG. 17. For this purpose, the targeting device 7 likewise comprises, at a laterally facing end of the crescent-shaped embodiment, an opening through which a master sleeve 8 can inserted. If an incision has been introduced in the thigh, tissue protection sleeves 9 which comprise visual openings 12 can be introduced through the master sleeve 8. The tissue protection sleeves 9 are positioned relative to one another at a slight angle of approximately 1° to 1.5°.

Once the tissue protection sleeves 9 have been introduced, the surgical treatment of a proximal femur fracture can subsequently take place. For this purpose, two drill wires 10, 11 are first introduced, wherein suitable guide sleeves for the drill wires 10, 11 are slid into the tissue protection sleeves 9 and the master sleeve 8. A positioning of the drill wires 10, 11 can be monitored via the visual openings 12. The guide sleeves 9 are thereby designed such that the superior drill wire 10 and the inferior drill wire 11 are introduced such that they run parallel to one another and are then arranged accordingly. A temporary, interoperational rotational stability is achieved with the drill wires 10, 11. Once the drill wires 10,11 have been introduced, namely into a femoral head for example, a rotation of the femoral head is no longer possible. After the introduction of the drill wires 10,11, the guide sleeve 9 for the superior drill wire 10 is removed and the bone screw 3 is introduced. The superior drill wire 10 can then be removed. The bone screw 3 is aligned such that a straight, lateral (visible in FIG. 15) guide line on the tool 6 is properly brought into agreement in line with a corresponding marking on the superiorly arranged tissue protection sleeve 9. The bone screw 3, which comprises one or more grooves 32 for the engagement of the set screw 4, should thus be correctly positioned. The inferior drill wire 11 is then removed and a guide sleeve for a drill for the set screw 4 is introduced. If the bone screw 3 is not in an ideal position, for example because a patient was repositioned or because the superiorly arranged tissue protection sleeve 9 became twisted prior to the alignment of the bone screw 3 at the marking due to other processes (whereby the bone screw 3 is then incorrectly aligned), this can be corrected by a slight rotation of the bone screw 3 using the tool 6, so that the drill can be laterally guided past the bone screw 3 as far as necessary. A similar procedure results if the set screw 4 should not be able to engage in a designated contact volume defined by a groove 32 of the bone screw 3, but rather already strikes the bone screw 3 beforehand in a direction viewed from lateral to medial, and thus in an undesired position. Once the set screw 4 has been inserted, an apposition and/or a compression can subsequently be performed in the previously described manner, if necessary.

It is noted that the foregoing examples have been provided merely for the purpose of explanation and are in no way to be construed as limiting of the present invention. While the present invention has been described with reference to an exemplary embodiment, it is understood that the words which have been used herein are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

What is claimed:

1. A tool for a set for treating bone fractures, in particular proximal femur fractures, comprising:
a base body having a first, in particular proximal, end and a second, in particular distal, end;
a grip region; and
a connector for positioning the tool on a part of the set, in particular a bone screw,
wherein the connector has a groove.

2. The tool according to claim 1, wherein the groove runs in a longitudinally extended manner between a lateral end and a medial end.

3. The tool according to claim 2, wherein the groove runs obliquely relative to a fastening axis.

4. The tool according to claim 1, wherein the connector is embodied with a shaft-shape.

5. The tool according to claim 1, wherein the connector, at a medial end, comprises a receptacle for the fitting-on and fastening of the tool to an, in particular lateral, end of a bone screw.

6. The tool according to claim 1, wherein the base body comprises an adjusting screw, which runs parallel to a fastening axis.

7. The tool according to claim 6, wherein the base body comprises an internal thread into which an external thread of the adjusting screw engages, which is embodied to be longer than a distance between a lateral edge and a medial edge of the base body in a region of the adjusting screw.

8. The tool according to claim 1, wherein the tool comprises a locking device for releasably fastening the tool to a further part of the set.

9. The tool according to claim 8, wherein the locking device is arranged in the grip region.

10. The tool according to claim 8, wherein the locking device is mounted such that it can be longitudinally displaced between the first end and second end.

11. The tool according to claim 8, wherein the tool comprises a release and fixing mechanism for the locking device.

12. The tool according to claim 8, wherein the locking device is mounted to be displaceable transversely to the fastening axis.

13. The tool according to claim 1, wherein the base body is embodied to be essentially flat.

14. A set for treating bone fractures, in particular proximal femur fractures, comprising:
a bone nail;
a bone screw;
a set screw; and
a tool for, in particular rotating, movement of the bone screw in a fastened state according to claim 1,
wherein the tool comprises a groove for the set screw,
wherein the bone nail comprises an opening through which the bone screw is guided, and
wherein the set screw is configured to be brought into contact with the bone screw in order to define at least one movement possibility of the bone screw.

15. The set according to claim 14, wherein the set screw is fixable in the bone nail such that the set screw runs at an angle oblique to the bone screw.

16. The set according to claim 14, wherein the set screw is configured to engage in a contact volume defined by the bone screw.

17. A method for treating bone fractures, in particular proximal femur fractures, in particular with a set according to claim 14, the method comprising:
guiding the bone screw and the set screw, which interacts with the bone screw, through an opening of the bone nail arranged in a bone piece, the set screw arranged to run obliquely to the bone screw;
defining movement possibilities of the bone screw, which includes at least one of bringing the set screw into contact with the bone screw; and
to position the set screw, moving the bone screw using the tool.

18. The method according to claim 17, further comprising rotatably moving the bone screw by rotation of the tool.

19. The method according to claim 17, further comprising grasping the bone screw using the tool; and
moving the bone screw in a lateral direction using an adjusting screw of the tool.

20. The method according to claim 17, further comprising grasping the bone screw using the tool;
positioning the tool at a stop one of connected to the set screw or formed by the set screw itself,
moving the bone screw in a lateral direction via a medially striking element of the tool.
